(19) United States
(12) Patent Application Publication
(10) Pub. No.: US 2025/0274690 A1
(43) Pub. Date: Aug. 28, 2025

(54) SIGNAL GENERATOR FOR AURICULAR CONCHAE

(71) Applicant: Hardcore Healing Inc., Lawrence Township, NJ (US)

(72) Inventors: Emily IRELAND, Lawrence Township, NJ (US); Eugene PALATNIK, Pewaukee, WI (US)

(73) Assignee: Hardcore Healing, Inc., Lawrence Township, NJ (US)

(21) Appl. No.: 18/942,586

(22) Filed: Nov. 9, 2024

Related U.S. Application Data
(63) Continuation-in-part of application No. 17/472,603, filed on Sep. 11, 2021, now abandoned.
Application No. 17/472,603 is a continuation-in-part of application No. 16/708,617, filed on Dec. 10, 2019, now Pat. No. 11,185,692.
(60) Provisional application No. 62/735,085, filed on Oct. 31, 2018.

(51) Int. Cl. H04R 1/02; H04R 1/10; H04R 3/00

(52) U.S. Cl. CPC H04R 1/028; H04R 1/1016; H04R 1/1033; H04R 1/1041; H04R 3/00

(57) ABSTRACT

A signal generator for auricular conchae includes an earpiece housing, a flange portion and a skin-contacting electrode element for contacting a concha of a user, where the skin contacting electrode element includes a coupling column with a longitudinal axis and a polygonal circumferential surface region.

### CROSS-REFERENCE TO RELATED PATENT DOCUMENTS

The present application is a Continuation-In-Part of U.S. patent application Ser. No. 17/472,603, filed Sep. 11, 2021; which is a Continuation-In-Part of U.S. patent application Ser. No. 16/708,617, filed, Dec. 10, 2019 and issued as U.S. Pat. No. 11,185,692 on Nov. 30, 2021; which claims the benefit of priority of U.S. Provisional Application No. 62/735,085 entitled “LIGHT TOUCH THERAPEUTIC HEADSET,” filed Oct. 31, 2018, the disclosures of all of the above being herein incorporated by reference in their entireties.

### FIELD OF THE INVENTION

The present application relates to signal generating devices and, more particularly, to a personal signal generator.

### SUMMARY

The physical condition of a human is reflected on the human's ears. It has been suggested that the skin resistance of small regions of the conchae differ from that of the immediate surroundings of the ear. These regions can be of a higher or lower resistance than their immediate surroundings. Points of high resistance are referred to as positive points and points of low resistance are referred to as negative points. It has been established that when the centers of positive points are inundated with electrical pulses having a higher potential at the centers of the points, the points return to resistive equibalance with the surroundings after a period of time.

It will be understood that this disclosure is not limited to the particular systems and methodologies described herein, as there can be multiple possible embodiments of the present disclosure which are not expressly illustrated in the present disclosure. It is also to be understood that the terminology used in the description is for the purpose of describing the particular versions or embodiments only and is not intended to limit the scope of the present disclosure.

In an embodiment, a wireless and self-contained headset delivers an electronic signal to a precise and self-locating position within bilateral conchae in both ears of a human user. The headset comprises an electronics housing that carries a specific waveform source. The waveform source generates the electronic signal, which is delivered simultaneously to both left and right ears of the human user. Each earpiece includes an electrode arranged to be disposed in contact with the relatively smooth plane of the conchae directly behind the external auditory meatus (ear opening/canal).

The device includes two earpiece housings. Where the housings include a waveform source for generating an electronic signal, the electronic signal is transmitted from the electronics housing and is communicated to a precise and self-locating position within bilateral conchae in each ear of the user. In certain embodiments, the device is made of a foam-like material that is self-fitting in the user's ear regardless of shape or size. In certain embodiments, the fit of the device in the ear is comfortable enough to be worn while sleeping and expands slightly to cover bilateral conchae. Certain embodiments of the device are shaped like an hourglass, such that a proximal section which touches conchae is wider than center, while a distal section which extends outward from the ear and holds the electrical housing is also wider than the center. The electrical frequency transmits through electrodes that touch skin of the bilateral conchae.

The user's device prepared according to principles of the invention may be more receptive to therapies and rehabilitative methods.

These and other features and advantages of the present invention will become apparent from the detailed description below, in light of the accompanying drawings.

### BRIEF DESCRIPTION OF THE DRAWINGS

The novel features which are believed to be characteristic of the present invention, as to its structure, organization, use and method of operation, together with further objectives and advantages thereof, will be better understood from the following drawings in which a presently preferred embodiment of the invention will now be illustrated by way of various examples. It is expressly understood, however, that the drawings are for the purpose of illustration and description only and are not intended as a definition of the limits of the invention. Embodiments of this invention will now be described by way of example in association with the accompanying drawings in which:

FIG. 1 shows, in schematic perspective view, a headset device prepared according to principles of the invention;

FIG. 2 shows, in functional block diagram form, the electronics of a headset device, prepared according to principles of the invention;

FIG. 3 shows, in schematic perspective view, the ears of an exemplary user of a device prepared according to principles of the invention, identifying conchal regions of the ears;

FIG. 4 illustrates, in schematic perspective view, the ears of an exemplary user and methods of use according to principles of the invention;

FIG. 5 shows, in schematic perspective view, an exemplary user and methods of use according to principles of the invention;

FIG. 6 in mechanical schematic view, a portion of an earpiece prepared according to principles of the invention;

FIG. 7 illustrates, in schematic perspective view, the ears of an exemplary user and methods of use according to principles of the invention;

FIG. 8 shows, in cutaway perspective view, further aspects of a signal generator headset prepared according to principles of the invention;

FIG. 9 shows, in cutaway perspective view, certain aspects of an electrode element for a signal generator headset;

FIG. 10 shows, in cutaway perspective view additional aspects of a housing for a signal generator headset;

FIG. 11A shows, in cutaway perspective view, certain aspects of an earpiece assembly for a signal generator headset prepared according to principles of the invention;

FIG. 11B shows, in cutaway perspective view, additional details of an electrode element for a signal generator headset prepared according to principles of the invention;

FIG. 11C shows, in cutaway perspective view, additional details of a flange portion for a signal generator headset prepared according to principles of the invention;

FIG. 11D shows, in cutaway perspective view, additional details of a housing portion for a signal generator headset prepared according to principles of the invention;

FIG. 12A-12H show, in mechanical schematic form, exemplary cross-sectional profiles of an electrode element support column for a signal generator headset prepared according to principles of the invention where:

FIG. 12A shows a triangular profile;

FIG. 12B shows a square profile;

FIG. 12C shows a pentagonal profile;

FIG. 12D shows a rectangular profile;

FIG. 12E shows a stellate profile;

FIG. 12F shows an irregular profile; and

FIG. 12G shows a compound profile;

FIG. 13A shows, in perspective view, control features for a signal generator headset earpiece prepared according to principles of the invention; and

FIG. 13B shows, in perspective cutaway view, a sealing element for a signal generator headset earpiece prepared according to principles of the invention.

### DETAILED DESCRIPTION

As used in the specification and claims, the singular forms “a”, “an”, and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention. There may be additional components described in the foregoing application that are not depicted on one of the described drawings. In the event such a component is described, but not depicted in a drawing, the absence of such a drawing should not be considered as an omission of such design from the specification.

The components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the present invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the present invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

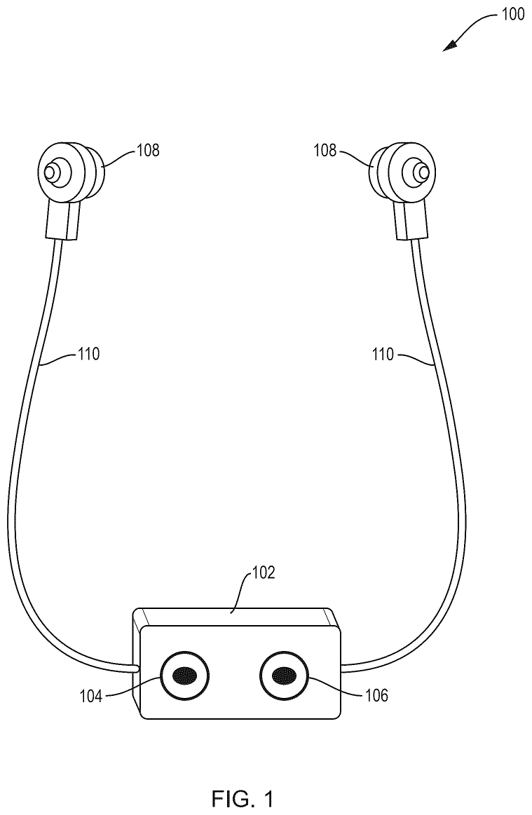

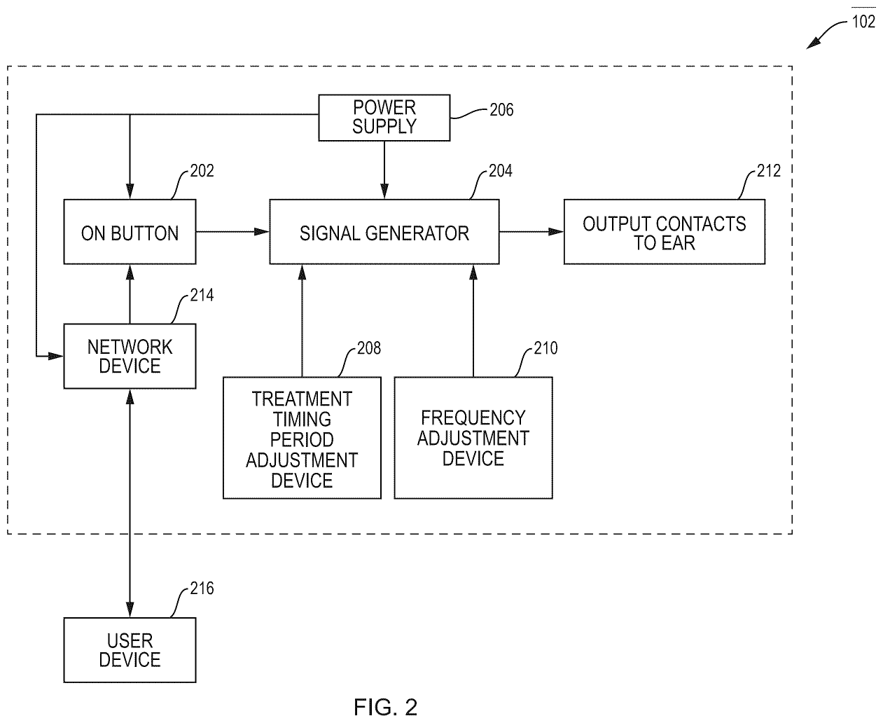

FIG. 1 is a diagram that illustrates a headset device 100, according to an exemplary embodiment of the present invention. The headset device 100 includes an electronics housing 102. The electronics housing 102 includes various components such as a power switch 104 and a wireless mode switch 106. The electronics housing 102 further includes a specific waveform source (e.g., a signal generator 204 shown in FIG. 2).

The waveform source generates an electronic signal, which is delivered simultaneously to both left and right ears of a human user. The headset device 100 further includes earpiece housings 108 and headset cables 110. Each headset cable 110 is a long piece that runs from the electronics housing 102 to each earpiece housing 108. The headset cables 110 is a set of wires coated in rubber or other nonconductive material. Its job is to act as a sort of bridge along which the electronic signal from the electronics housing 102 may travel up to each earpiece housing 108.

In an embodiment, the electronics housing 102 carries the waveform source device in communication with right and left tissue interface circuits, carried respectively in right and left earpiece housings 108. The electronics housing 102 carries the waveform source device having an impedance detecting function. The right and left earpiece housings 108 are each connected to the electronics housing 102 by means of the right and left headset cables 110, respectively, and are carried in suitable positions for application, respectively, to the right and left ears of the user. The right and left earpiece housings 108 include right and left elongated protrusions, respectively, and each extending to a respective free end wall from the right and left earpiece housings 108.

In an embodiment, the earpiece housings 108 carry a tissue interface circuit on the free end wall, and the tissue interface circuits are in communication with the waveform source device for communicating impedance and receiving treatment signals such as the electronic signals generated by the waveform source device (i.e., the signal generator 204 shown in FIG. 2). Also, the elongated protrusions are suitably arranged for applying the respective tissue interface circuits against the concha of the ear of the user when the headset device 100 is applied to the user. In an embodiment, the tissue interface circuit comprises an array of electrodes carried in association with the free end wall of each earpiece housing 108 and is sized to typically contact at least about one-quarter the height of the concha of the user's ear. The array is arranged to achieve electrical communication with the preselected contact areas.

In an embodiment, the wireless and self-contained headset device 100 delivers the electronic signal to a precise and self-locating position within bilateral conchae in both ears of the user who is wearing the headset device 100. The headset device 100 comprises the electronics housing 102 that carries the specific waveform source. The waveform source generates the electronic signal, which is delivered simultaneously to both left and right ears of the user. An array of silver electrodes housed within the bilateral earpieces cover with precision the location at the exact center of the relatively smooth plane of the conchae directly behind the external auditory meatus (ear opening/canal). The waveform source generates the electronic signal for which variability or range of frequency does not change i.e., the frequency of the electronic signal is invariable and never wavers but is constant. In an embodiment, the constant or invariable frequency level of the electronic signal is set at 90 Hz and does not change. In some examples, the constant or invariable frequency level of the electronic signal may be set at a frequency in a range of 70-110 cycle per second (cps) or Hz.

Further, the waveform source is unmodulated and does not change. Further, the electronic signal is a square pulse waveform that is unmodulated. In an embodiment, there are not any auditory signals, or sounds of any kind, such as music or tones, emitted from this headset device 100.

The headset device 100 may be used by anyone who can pick up the device and place it on the ears, or if the case may be, on one's own head, with little instruction other than to turn it ON and place it upon the head of the user. In an embodiment, the headset device 100 is completely self-locating, unlike previously made devices, which require minimal to extensive training to use with successful results. This device does not require any preparation, knowledge or skills other than the ability to place it on the head of the user in need of help, and to turn the device's power switch to the ON position. Because it is a self-locating mechanism, anyone can use it with accuracy. In an embodiment, the recommended amount of time for usage is 40 minutes per day for a duration of 10 days, although it may also be used overnight, and is designed comfortable enough to be worn during sleep, or for greater amounts of time. The headset device 100 may be used with significant observable results within the first 30 minutes. There is no maximum treatment time as the device can be worn up to 6 hours per day or longer, even to sleep in.

The illustrated apparatus includes a self-contained, portable, and wireless headset device 100 that carries the selectively activated waveform source device that causes the electronic signal responsive to the measured impedance. The right and left earpiece housings 108 each carry the tissue interface circuit that is responsive to the source device to deliver unmodulated square pulse waveform. The tissue interface circuit is configured with an ear-entering portion that is suitably sized and shaped for application onto the concha of the user's ear. The ear-entering portion has a free end that carries a contact portion of the tissue interface circuit.

The contact portion is an array of electrodes suitably sized and shaped for contacting the concha of the user's ear and, specifically, for contacting a preselected contact area near the lower edge of the ear canal opening and extending rearwardly from the canal. The interface circuits are applied to the ears of the user, in a position such that the tissue interface circuit is in communication with the preselected contact area (i.e., the precise and self-locating position within the bilateral conchae in both ears of the user). The source device may be activated by means of the power switch 104 to provide the constant or invariable electrical signal to the tissue interface circuits. As a result, the tissue interface circuits deliver an effective waveform treatment (by means of the unmodulated square pulse electronic signal that is set at any frequency in a range of 70-110 Hz, preferably at 90 cps or 90 Hz).

In some embodiments, the user may activate the source device by using an application running on a user device (e.g., a smartphone) of the user. The user device may wirelessly communicate with the headset device 100 when the user activates the wireless mode switch 106. Based on such activation, the user device and the headset device 100 may communicate over a communication network. When communicatively connected, the user may control ON-OFF of the headset device 100 by means of the user device. For example, the user, by means of the application running on the user device, may select an option to turn ON the waveform source (e.g., a signal generator 204 shown in FIG. 2). Based on the selection, the user device may communicate a turn ON signal to the waveform source device over the communication network. Accordingly, the waveform source device may receive the turn ON signal from the user device and generates the unmodulated square pulse electronic signal at a constant or invariable frequency in a range of 70-110 Hz, preferably at 90 cps or 90 Hz for facilitating electrotherapy.

Examples of types of the communication network may include, but are not limited to, a local area network, a wide area network, a radio network, a virtual private network, an internet area network, a metropolitan area network, a satellite network, Wi-Fi, Bluetooth Low energy, a wireless network, and a telecommunication network. Examples of the telecommunication network may include, but are not limited to, a global system for mobile communication (GSM) network, a general packet radio service (GPRS) network, third Generation Partnership Project (3GPP), an enhanced data GSM environment (EDGE), and a Universal Mobile Telecommunications System (UMTS).

FIG. 1 shows a front view of the headset device 100 that demonstrates the position in which the headset is usually worn by the user, although it may be designed in the reverse position, wrapped around the top of the head like a band as a more typical headphone set would normally be worn. However, in the position shown, it is worn hanging underneath the head and chin, fitted like a stethoscope. Sizing is adjustable to fit any user of any size or age. The electronics housing 102, which carries the waveform source, is held within the headset band.

FIG. 2 is a block diagram that illustrates the electronics housing 102 of the headset device 100, according to an exemplary embodiment of the present invention. The electronics housing 102 includes an ON button 202, a signal generator 204, a power supply 206, a treatment timing period adjustment device 208, a frequency adjustment device 210, and output contacts 212 to the user's ear. The electronics housing 102 may further include a network device 214 for facilitating communication with a user device 216 of the user.

In an embodiment, the headset device 100 may be turned ON by turning on the ON button 202 by means of the power switch 104. The signal generator 204 is turned on with power supplied by the power supply 206 (such as a battery). The signal generator 204 generates the electronic signal for which variability or range of frequency does not change i.e., the frequency of the electronic signal is invariable and never wavers but is constant. In an embodiment, the constant or invariable frequency level of the electronic signal is set at a frequency of 90 Hz and does not change. Further, the waveform source is unmodulated and does not change.

Further, in certain embodiments, the electronic signal is a square pulse waveform that is unmodulated. In an embodiment, there are not any auditory signals, or sounds of any kind, such as music or tones, emitted from this headset device 100. The signal generator 204 transmits the electronic signal through the output contacts 212 to the user's ear. In an embodiment, the pattern of treatment using the constant electronic signal is adjusted for duration of treatment by means of the treatment timing period adjustment device 208 to deliver the constant or invariable frequency level of the electronic signal set at 90 Hz over the set time periods.

In an embodiment, the recommended amount of time for usage is 40 minutes per day for a duration of 10 days, although it may also be used overnight, and is designed comfortable enough to be worn during sleep, or for greater amounts of time. The headset device 100 may be used with significant observable results within the first 30 minutes. There is no maximum treatment time as the device can be worn up to 6 hours per day or longer, even to sleep in. In an embodiment, the frequency adjustment device 210 sets the generation of the electronic signal at 90 Hz by means of the signal generator 204.

In an embodiment, the network device 214 facilitates wireless communication between the user device 216 and the headset device 100. The user device 216 may wirelessly communicate with the headset device 100 when the user activates the wireless mode switch 106 to turn ON the network device 214 that facilitates the wireless communication between the user device 216 and the headset device 100. Based on such activation, the user device 216 and the headset device 100 may communicate over the communication network. When communicatively connected, the user may control ON-OFF of the headset device 100 by means of the user device 216. For example, the user, by means of the application running on the user device 216, may select an option to turn ON the signal generator 204. Based on the selection, the user device 216 may communicate a turn ON signal to the signal generator 204 over the communication network. Accordingly, the signal generator 204 may receive the turn ON signal from the user device 216 and generate the unmodulated square pulse electronic signal at a constant or invariable frequency of 90 cps or 90 Hz for facilitating electrotherapy.

Examples of types of the communication network may include, but are not limited to, a local area network, a wide area network, a radio network, a virtual private network, an internet area network, a metropolitan area network, a satellite network, Wi-Fi, Bluetooth Low energy, a wireless network, and a telecommunication network. Examples of the telecommunication network may include, but are not limited to, a global system for mobile communication (GSM) network, a general packet radio service (GPRS) network, third Generation Partnership Project (3GPP), an enhanced data GSM environment (EDGE), and a Universal Mobile Telecommunications System (UMTS).

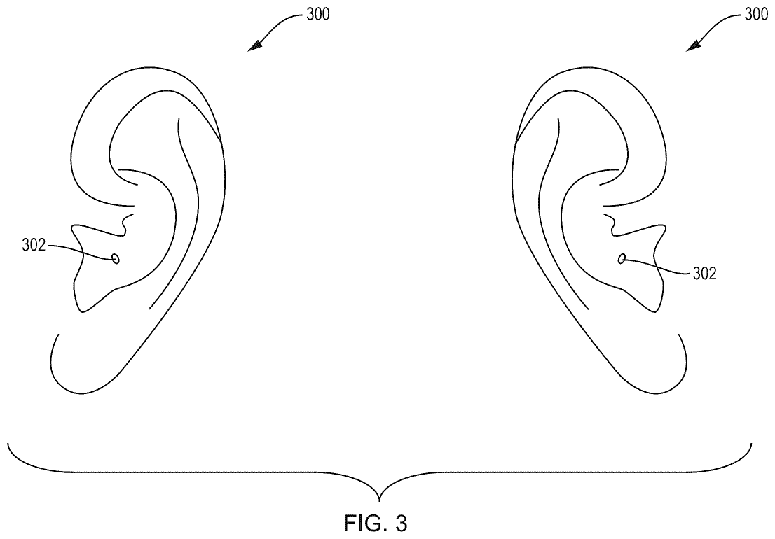

FIG. 3 is a diagram 300 that illustrates a precise location area 302 in an ear of the user, according to an exemplary embodiment of the present invention. FIG. 3 demonstrates the precise location area 302 desired for the treatment. To locate on a human ear, find the smooth plane of the ear called the concha, which is located directly posterior to, or behind, each ear opening (external auditory meatus). It is firm or hard when palpated, much like the skull when pressed with a finger, rather than the fleshier, softer parts of the ear. The location specified aligns precisely behind the center of the ear opening, directly in the center of the bilateral conchae.

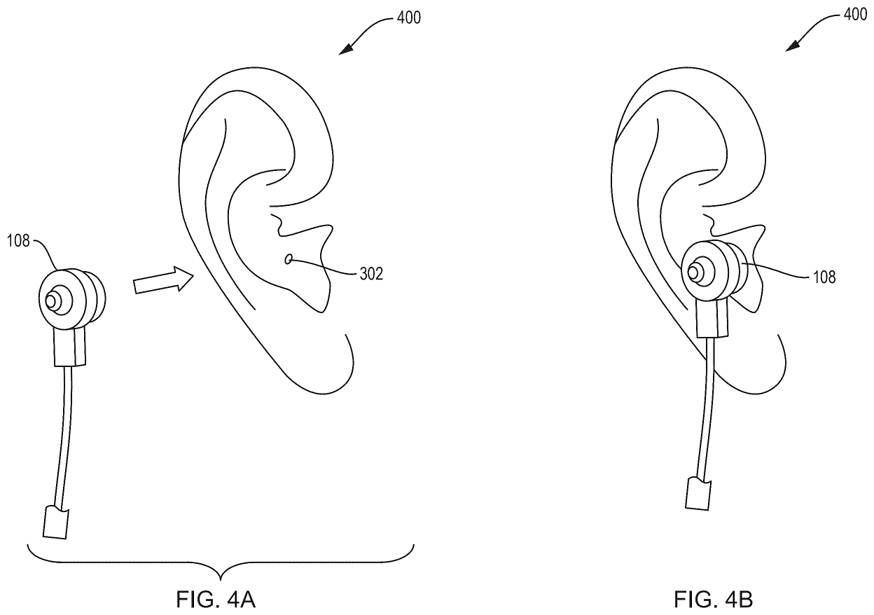

FIG. 4 is a diagram 400 that illustrates an exemplary scenario for inserting the headset device 100 into the precise location area 302 in the ear of the user desired for the treatment, according to an exemplary embodiment of the present invention. In an embodiment, each earpiece housing 108 is inserted into the precise location area 302 in the ear of the user. The location specified aligns precisely behind the center of the ear opening, directly in the center of the bilateral conchae.

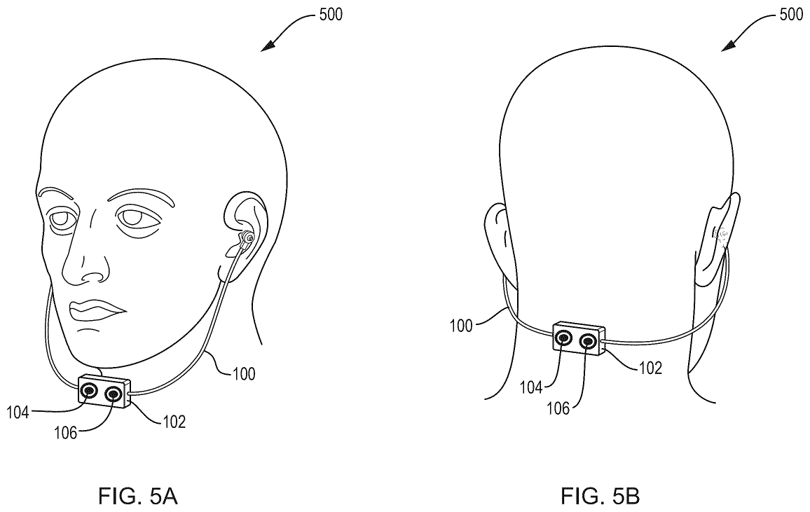

FIG. 5 is a diagram 500 that illustrates an exemplary scenario of the user with the headset device 100 into the precise location area 302 in the ear of the user, according to an exemplary embodiment of the present invention. The headset device 100 may be worn over the head or around the user's neck hanging either in the front or rear portion of the user's head. The user may be more receptive to other therapies as well as rehabilitative methods.

According to one embodiment of the invention, it is recommended to use the device 40 minutes per day for 10 days. There is no maximum treatment time as the device can be worn up to 6 hours per day or longer, even to sleep in. Additional benefits include increased receptivity to and enhancement of psychotherapy, counseling, and other rehabilitative methods. The return to a normal sleep pattern, which also enhances healthy physiological functioning, mood, and rehabilitation. Improved self-esteem and mental health, including a positive outlook on life and emotional well-being. Improved motivation and ability to successfully manage daily life activities independently. Improved communication skills, comprehension, calmness and clarity of the mind. Improved physical coordination. Enhanced self-awareness. A marked decrease in depression and anxiety levels.

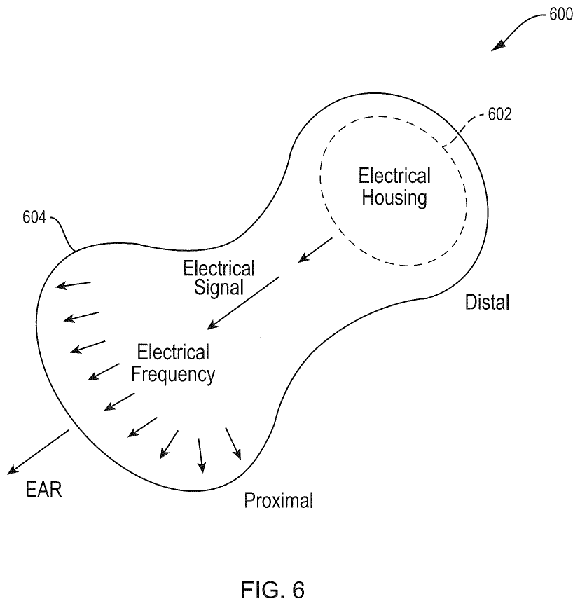

FIG. 6 is a diagram that illustrates an earpiece device 600, according to an exemplary embodiment of the present invention. The earpiece device 600 includes an electronics housing 602 as shown in the FIG. 6. The electronics housing 602 may be similar to the one shown in FIGS. 1 and 2 and may communicate wirelessly with one or more devices such as a user device (like smartphone) of the user. The electronics housing 602 may be located in the distal location of the earpiece housing 604. The electronics housing 602 may include various components such as a wireless mode switch and an input sensing element. The electronics housing 602 further includes a waveform source (e.g., the signal generator 204 shown in FIG. 2) that is configured to generate an electronic signal, which is delivered simultaneously to both left and right ears of a human user when the user has put on left and right earpieces into the left and right ears.

In an embodiment, the earpiece device 600 is a wireless earpiece that is made of a foam-like material (for example, any type of soft, form-fitting material that molds to the natural form of the body) that is self-fitting in anyone's ear, regardless of size or shape. The fit of the in the ear is comfortable enough to be worn while sleeping and expands slightly to cover bilateral conchae. Each (i.e., left or right earpiece) is shaped like an hourglass such that a proximal section (as shown) which touches the conchae is wider than the center, while the distal section (as shown) which extends outward from the ear (and which holds the electrical housing/information) is also wider than the center. The electrical frequency transmits through electrodes that touch the skin of the bilateral conchae. However, if a needle array is used for the electrical current, these needles do not penetrate through the skin.

In an embodiment, the electronics housing 602 carries the waveform source device in communication with right and left tissue interface circuits, carried respectively in right and left earpieces. The electronics housing 602 carries the waveform source device having an impedance detecting function. The right and left earpieces include right and left elongated protrusions, respectively, and each extending to a respective free end wall from the right and left earpiece housings 604.

In an embodiment, the earpiece housing 604 may carry a tissue interface circuit on the free end wall, and the tissue interface circuits are in communication with the waveform source device for communicating impedance and receiving treatment signals such as the electronic signals generated by the waveform source device (i.e., the signal generator 204 shown in FIG. 2). Also, the elongated protrusions are suitably arranged for applying the respective tissue interface circuits against the concha of the ear of the user when the earpiece device 600 is applied to the user. In an embodiment, the tissue interface circuit comprises an array of electrodes carried in association with the free end wall of each earpiece housing 604 and is sized to typically contact at least about one-quarter the height of the concha of the user's ear. The array is arranged to achieve electrical communication with the preselected contact areas.

In an embodiment, the wireless and self-contained earpiece device 600 (i.e., the left or right earpiece) may be configured to deliver the electronic signal to a precise and self-locating position within bilateral conchae in both ears of the user who is wearing both of the left and right earpieces. The earpiece device 600 includes the electronics housing 602 that carries the specific waveform source. The waveform source generates the electronic signal, which is delivered to the respective ear of the user. An array of silver electrodes housed within the bilateral earpieces cover with precision the location at the exact center of the relatively smooth plane of the conchae directly behind the external auditory meatus (ear opening/canal). In one embodiment, the waveform source generates the electronic signal for which variability or range of frequency does not change i.e., the frequency of the electronic signal is invariable and never wavers but is constant. In an embodiment, the constant or invariable frequency level of the electronic signal may correspond to any frequency in a range from at least about 70 hertz to at least about 110 hertz.

In one preferred exemplary scenario, the constant or invariable frequency level of the electronic signal is set at 90 Hz and does not change. Further, the waveform source is unmodulated and does not change. Further, the electronic signal is a square pulse waveform that is unmodulated. In an embodiment, there are not any auditory signals, or sounds of any kind, such as music or tones, emitted from this earpiece device 600. In an embodiment, the earpiece device 600 does not require any type of training to use. The earpiece device 600 may be used by anyone who can pick up the device and place it on the ear with little instruction other than to turn it ON and place it into the ear of the user who needs treatment. In an embodiment, the earpiece device 600 is completely self-locating, unlike previously made devices, which require minimal to extensive training to use with successful results.

This device does not require any preparation, knowledge or skills other than the ability to place it into the ear of the user in need of help, and to turn the device's power to the ON position. Because it is a self-locating mechanism, anyone can use it with accuracy. In an embodiment, the recommended amount of time for usage is 40 minutes per day for a duration of 10 days, although it may also be used overnight, and is designed comfortable enough to be worn during sleep, or for greater amounts of time. The earpiece device 600 may be used with significant observable results within the first 30 minutes. There is no maximum treatment time as the device can be worn up to 6 hours per day or longer, even to sleep in.

The right and left earpiece housings 604 each carry the tissue interface circuit that is responsive to the source device to deliver unmodulated square pulse waveform. The tissue interface circuit is configured with an ear-entering portion that is suitably sized and shaped for application onto the concha of the user's ear. The ear-entering portion has a free end that carries a contact portion of the tissue interface circuit. The contact portion is an array of electrodes suitably sized and shaped for contacting the concha of the user's ear and, specifically, for contacting a preselected contact area near the lower edge of the ear canal opening and extending rearwardly from the canal.

The interface circuits are applied to the ears of the user, in a position such that the tissue interface circuit is in communication with the preselected contact area (i.e., the precise and self-locating position within the bilateral conchae in both ears of the user). The source device may be activated by means of an input (such as a touch-based input, a voice-based input, or an app-based input provided by the user i.e., the wearer of the earpieces) to provide the constant or invariable electron signal to the tissue interface circuits. As a result, the tissue interface circuits deliver an effective waveform treatment (by means of the unmodulated square pulse electronic signal that is set at 90 cps or 90 Hz, (or to some other frequency in the range of e.g., 70-120 Hz).

In some embodiments, the user may activate the source device by using an application running on the user device (e.g., a smartphone) of the user. The user device may wirelessly communicate with the earpiece device 600. Based on such activation, the user device and the earpiece device 600 may communicate over a communication network. When communicatively connected, the user may control ON-OFF of the earpiece device 600 by means of the user device or by means of the touch-based input or the voice-based input. In one example, the user, by means of the application running on the user device, may select an option to turn ON the waveform source (e.g., a signal generator 204 shown in FIG. 2).

Based on the selection, the user device may communicate a turn ON signal to the waveform source device over the communication network. Accordingly, the waveform source device may receive the turn ON signal from the user device and generates the unmodulated square pulse electronic signal at a constant or invariable frequency of 90 cps or 90 Hz (or to some other frequency in the range of 70-110 Hz). Examples of types of the communication network may include, but are not limited to, a local area network, a wide area network, a radio network, a virtual private network, an internet area network, a metropolitan area network, a satellite network, Wi-Fi, Bluetooth Low energy, a wireless network, and a telecommunication network. Examples of the telecommunication network may include, but are not limited to, a global system for mobile communication (GSM) network, a general packet radio service (GPRS) network, third Generation Partnership Project (3GPP), an enhanced data GSM environment (EDGE), and a Universal Mobile Telecommunications System (UMTS).

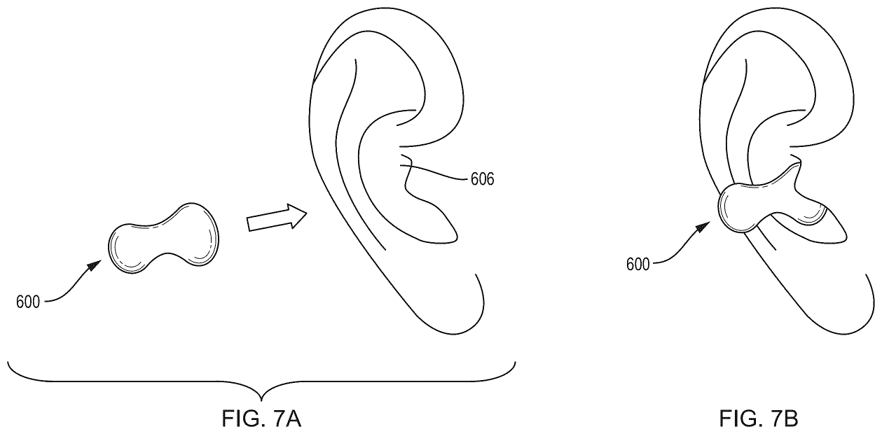

FIG. 7 is a diagram that illustrates an exemplary scenario for inserting the earpiece device 600 into the precise location area in the ear of the user desired for treatment, according to an exemplary embodiment of the present invention. In an embodiment, each earpiece device 600 is inserted into the precise location area 606 in the ear of the user. The location specified aligns precisely behind the center of the ear opening, directly in the center of the bilateral conchae. The wireless is made of a foam-like material (any type of soft, form-fitting material that molds to the natural form of the body) that is self-fitting in anyone's ear, regardless of size or shape. The fit is comfortable enough to be worn while sleeping and expands slightly to cover bilateral conchae.

The present invention discloses the headset device 100 or the earpiece device 600. In operation, the user inserts the earpieces into the precise location area in the user's ear. Thereafter, the headset device 100 or the earpiece device 600 is switched ON. Constant unmodulated electronic signal at 90 Hz frequency (or at some other frequency in the range of 70-110 Hz) is delivered to bilateral concha region to stimulate the temporal bones and related attachments, including the articulation with the sphenoid bone, the cranial sutures and mobility of the cranial bones as they interrelate, the choroid plexus and all structures related to the cerebrospinal fluid pump mechanism, and intrinsic connective tissue. Once the user is done with an application, the device may be turned off, recharged, or otherwise put away until the next use is completed. User may be sitting, lying down, standing or otherwise ambulatory in any functional activity during treatment, although for best results a restful state is recommended.

FIG. 8 shows, in perspective view, a further earpiece device 800 prepared according to principles of the invention. In contrast to device 100 of FIG. 1, device 800 does not include a discrete electronics housing 102. Rather, all of the electronics of device 800 are contained within the earpiece housings 802 and 804 respectively. Accordingly, earpiece housing 802 and 804 are directly operatively coupled to one another at respective ends 806, 808 of an electrical cable 810.

Earpiece housings 802, 804 support respective skin-contacting electrode elements 812 and 814. In one exemplary embodiment, one of earpiece housings 802, 804 contains an electrical battery and charging and/or regulating circuit. The other earpiece housing includes the balance of the electronic signal generator circuitry. In such an embodiment, cable 810 includes a plurality of electrical conductors including first 816 and second 818 electrical conductors.

In one embodiment of the invention, the electrical conductors 816, 818 serve to conduct both power and operative signal between the earpiece housings 802 and 804. Thus, in one embodiment, one conductor 816 serves as a common or ground conductor while the other conductor 818 completes a power circuit between the battery and signal generator while also providing a reference potential for an operative signal produced by the signal generator. In such an embodiment, for example, common electrical conductor 816 might be coupled, directly or indirectly, at end 806 to a negative terminal of a battery disposed within housing 802 and, in parallel with that connection, to skin-contacting electrode 812.

At end 808, the same electrical conductor 816 might be coupled to a common input power connection of a signal generator circuit disposed within earpiece housing 804. A positive terminal of the battery disposed within housing 802 would be coupled, directly or indirectly, at end 806 of the cable 810 to conductor 818, which would be connected to a power input terminal of the signal generator circuit disposed within earpiece housing 804. An output terminal of the signal generator disposed within earpiece housing 804 would be electrically coupled to skin-contacting electrode element 814, consequently providing a time-varying electrical signal between skin-contacting electrode 812 and skin-contacting electrode 814 during operation of the apparatus.

One of skill in the art will appreciate that, in an alternative embodiment of the invention, cable 810 will include, for example, four separate conductors. In such an arrangement, two conductors provide a power connection between a battery, or other electrical power source, in one earpiece housing and signal generator circuitry in the other. The third and fourth conductors will provide a signal path between the signal generator disposed within one earpiece housing and the skin-contacting electrode coupled to the other housing.

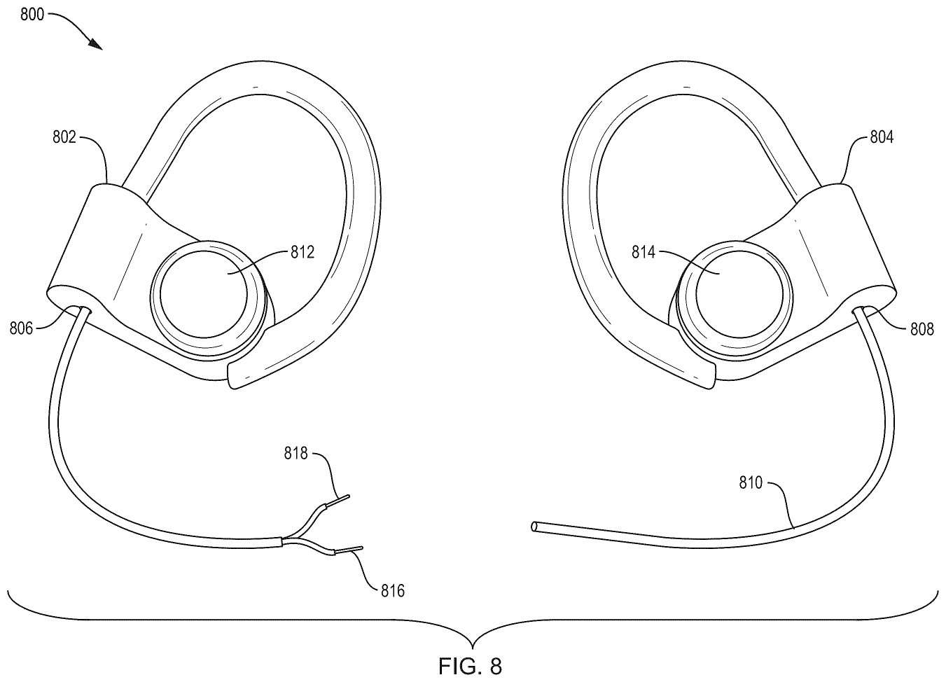

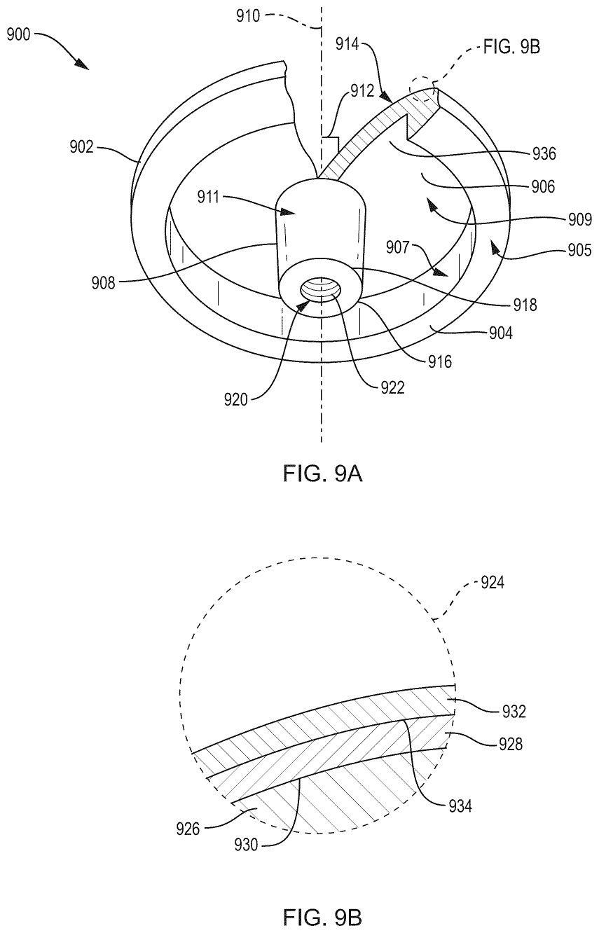

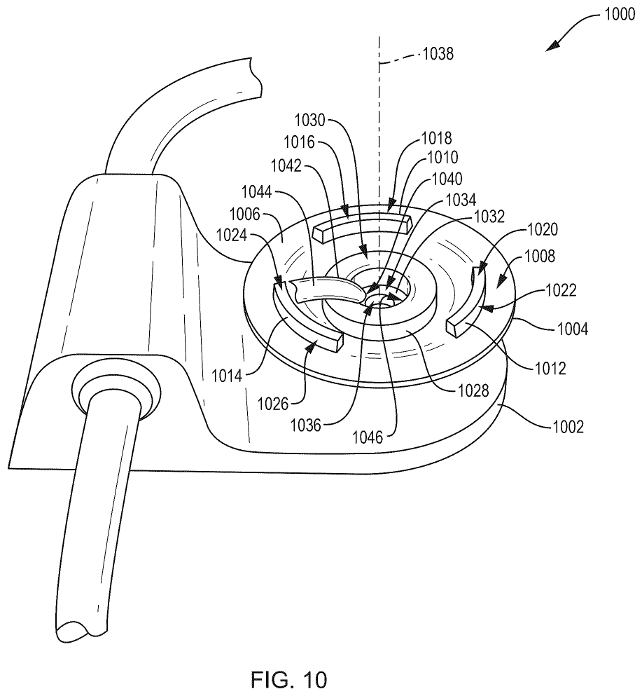

FIGS. 9 and 10 show one arrangement of a housing and skin-contacting electrode, including details of the structural and electrical connections of the same. Accordingly, FIG. 9 shows, in cutaway perspective view, an exemplary skin-contacting electrode element 900. The skin-contacting element 900 will include, for example, a body 902 with a first generally annular region 904, a structural portion 906, and a coupling column 908. In the example illustrated, the coupling column 908 is shown as a generally cylindrical solid having a circumferential surface region 911 and longitudinal axis 910 disposed generally normal 912 to skin-contacting surface region 914 of the structural portion 906. Annular region 904 exhibits first and second bearing surface regions 905 and 907. Structural portion 906 exhibits a second bearing surface region 909.

The illustrated coupling column 908 has a circumferential edge region 916 having a generally circular aspect. In certain arrangements, the circumferential edge region (and a corresponding transverse section) of the coupling column 908 includes a flattened region 918 such that a portion of the circumferential edge replaced by a chord of the circle, or such that the circumferential edge region 916 is generally elliptical or oblate.

In certain arrangements, the coupling column 908 includes a longitudinal bore 920. In certain embodiments of the invention, longitudinal bore 920 includes a plurality of internal threads 922 adapted to receive therewithin, for example, a machine screw. In other arrangements, longitudinal bore 920 includes a surface adapted to receive a self-threading fastener such that internal threads are formed by insertion of the fastener. In still other arrangements, longitudinal bore 920 will the arranged and configured to receive any of a variety of other fasteners such as are known, or become known, in the art.

In the illustrated arrangement, the skin-contacting electrode element 900 has an electrically conductive surface. Accordingly, as shown in detail view 924, body 902 of the skin-contacting electrode 900 includes, for example, a synthetic polymer material 926. A layer of electroless nickel material 928 disposed upon, and supported by, a surface region 930 of the polymer material 926. A further layer of electrolytically deposited silver 932 is disposed upon, and supported by, a further surface region 934 of the electrolytically deposited nickel material 928.

One of skill in the art will appreciate that the silver-over-nickel combination described above is merely exemplary of a wide variety of possible structures and arrangements beneficially employed in respective embodiments of the invention. Accordingly, in certain embodiments of the invention, a surface layer of the invention will include any conductive metallic or metallic alloy material such as, for example, a gold material, a platinum material, a stainless steel material, a titanium material, and combinations thereof. In other embodiments of the invention, the electrode element will include a layer of an electrically conductive polymer material such as, for example, an intrinsically conductive polymer material; a carbon filled polymer material; a metal filled polymer material, and combinations thereof.

In certain embodiments of the invention, a conductive layer will be applied to a substrate material as, for example, a liquid coating having an electrically conductive material, a binder and a volatile carrier, where the volatile carrier is allowed and/or caused to evaporate after application. The resulting deposit of conductive material is bound to the substrate providing a conductive path to the concha of a user.

In certain additional embodiments of the invention, the body of the electrode element is made entirely of, or includes, a conductive material such as, for example, any of those discussed above. Thus, the conductive material is a bulk material constituent of the body. Accordingly, in certain embodiments of the invention, no coating or additional layers of electrically conductive material are employed.

It will be appreciated that electrically conductive materials applicable in the present invention will preferably be biocompatible and may include, for example and without limitation, one or more of Alumina, Bioglass, Cobalt-chromium alloy, Hydroxyapatite, silicone polymer, Polyvinylchloride polymer, Polyethylene polymer, Polypropylene polymer, Polytetrafluoroethylene polymer, Polymethylmethacrylate polymer, Stainless steel, Trimethylcarbonate polymer, TMC NAD-lactide polymer, Titanium & titanium alloys, and Zirconia, and combinations thereof. In addition, conformal materials such as polymer foam materials and polymer-elastomer materials, as well as any material that may conform desirably to a surface topology of the electrode contact region, will be beneficially employed in certain embodiments of the invention

It will be appreciated by one of skill in the art that, as further discussed below, the electrically conductive nickel 928 and silver 932 materials will advantageously cover the entirety of the external surface of the skin-contacting electrode element 900. Accordingly, for example, a surface region 936 of structural portion 906, disposed in generally parallel spaced relation to skin-contacting surface region 914 is, in certain embodiments, fully covered by the nickel and silver materials. In light of the continuity of the layers of material 928 and 932, electrical continuity will exist between surface region 936 and skin-contacting surface region 914.

FIG. 10 shows further aspects of an exemplary earpiece housing 1000. As shown, earpiece housing 1000 includes a body portion 1002 with a flange portion 1004. The illustrated flange portion 1004 is integrally formed with the body portion 1002.

As shown, flange portion 1004 includes an upper surface region 1006. Upper surface region 1006 includes a circumferential surface region 1008. Considering together FIGS. 9 and 10, one of skill in the art will appreciate that circumferential surface region 1008 is well adapted to be disposed in contact with, and support, bearing surface region 905 of skin-contacting electrode element 900.

As illustrated, flange portion 1004 includes three arcuate stanchion portions 1010, 1012, 1014. The arcuate stanchion portions 1010, 1012 and 1014 have respective bearing surface regions 1016, 1018, 1020, 1022, 1024 and 1026. Considering together FIGS. 9 and 10, one of skill in the art will appreciate that bearing surface regions 1016, 1020 and 1024 are well adapted to be disposed in contact with, and support, respective portions of bearing surface region 909 of skin-contacting electrode element 900. Correspondingly, bearing surface regions 1018, 1022 and 1026 are well adapted to be disposed in contact with, and support, respective portions of bearing surface region 907 of skin-contacting electrode element 900.

As illustrated, flange portion 1004 includes a further generally annular stanchion portion 1028. The arcuate stanchion portion 1028 has a bearing surface region 1030. Considering together FIGS. 9 and 10, one of skill in the art will appreciate that bearing surface region 1030 is well adapted to be disposed in contact with, and support, a respective portion of bearing surface region 909 of skin-contacting electrode element 900.

One of skill in the art will appreciate that, in respective instances, stanchion portions 1010, 1012 and 1014 will be integrally formed with flange portion 1004. In other instances, one or more of the stanchion portions will be prepared as discrete elements and mechanically coupled to, or otherwise supported by, flange portion 1004.

As illustrated, flange portion 1004 includes a central region 1032 disposed inwardly of annular stanchion portion 1028. Central region 1032 has a surface region 1034. A first bore, or through-hole, 1036 is provided between surface region 1034 and an internal cavity of earpiece housing 1000. First bore 1036 is arranged to receive a fastener, such as a machine screw, self-threading screw, or other fastener, therethrough. In use, a longitudinal axis 1038 of bore 1036 is disposed coincident with longitudinal axis 910 of skin-contacting electrode element 900 so that the fastener can be disposed through bore 1036 into longitudinal bore 920 so as to substantially fixedly couple skin-contacting electrode element 900 to earpiece housing 1000.

In the illustrated arrangement, central region 1032 also includes a second bore 1040 between surface region 1034 and the internal cavity of earpiece housing 1000. The second bore 1040 is configured to receive a terminal portion 1042 of an electrical conductor therethrough.

As illustrated, surface region 1044 of terminal portion 1042 is uninsulated, and is disposed adjacent bearing surface region 1030. Consequently, when skin-contacting electrode element 900 is assembled to earpiece housing 1000, terminal portion 1042 is captured between surface region 1030 and a corresponding portion of surface region 909. This serves to mechanically retain terminal portion 1042 in place, and to place surface region 1044, and therefore the corresponding conductor, in electrical contact with the layer of conductive silver 932. Accordingly, an operative electrical coupling is formed between the electrical conductor and the skin-contacting portion of skin-contacting electrode 900.

One of skill in the art will appreciate that the external circumferential surface region 911 of coupling column 908 will, when assembled, be disposed adjacent to an internal surface region 1046 of annular stanchion 1028, where both the external surface region 911 and internal surface region 1046 have a generally circular cylindrical aspect.

To the extent beneficial in a particular instance, the presence of, e.g., a flattened region 918 provides clearance to accommodate the presence of terminal portion 1042, as well as additional surface area for establishing electrical communication between the conductor and the skin-contacting electrode.

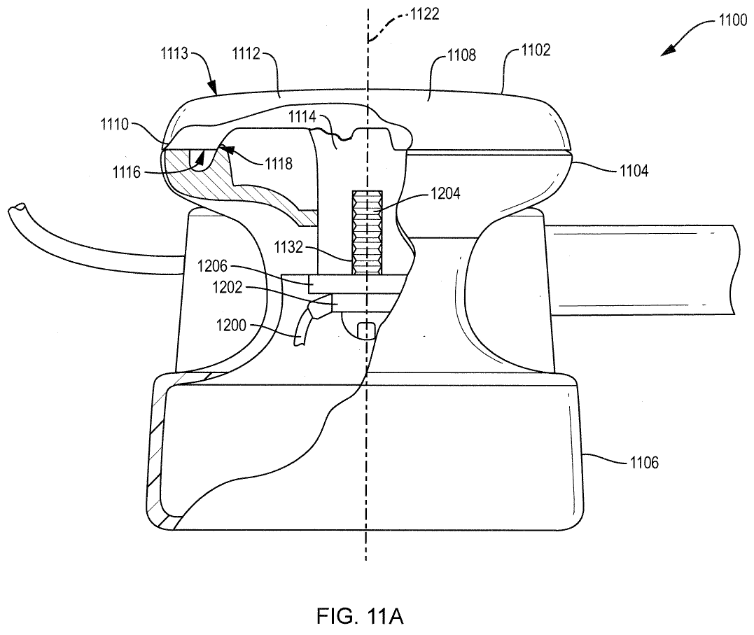

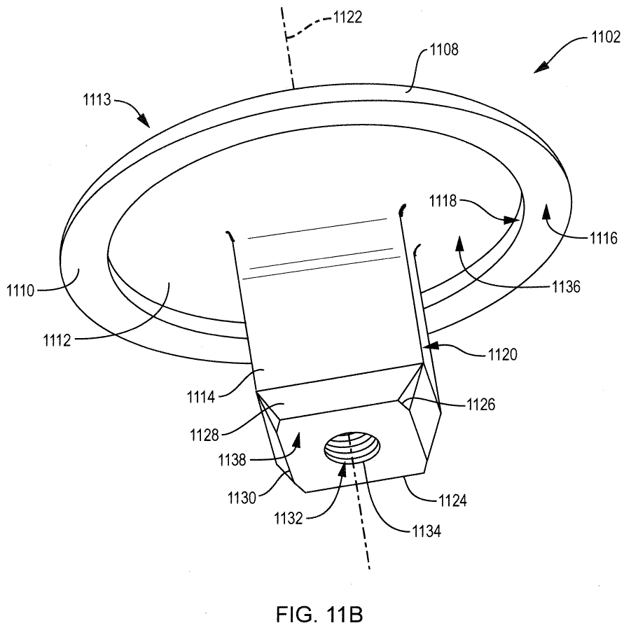

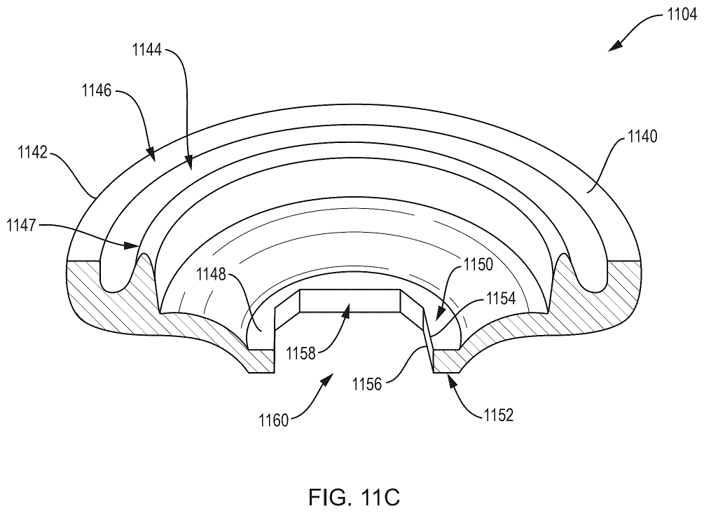

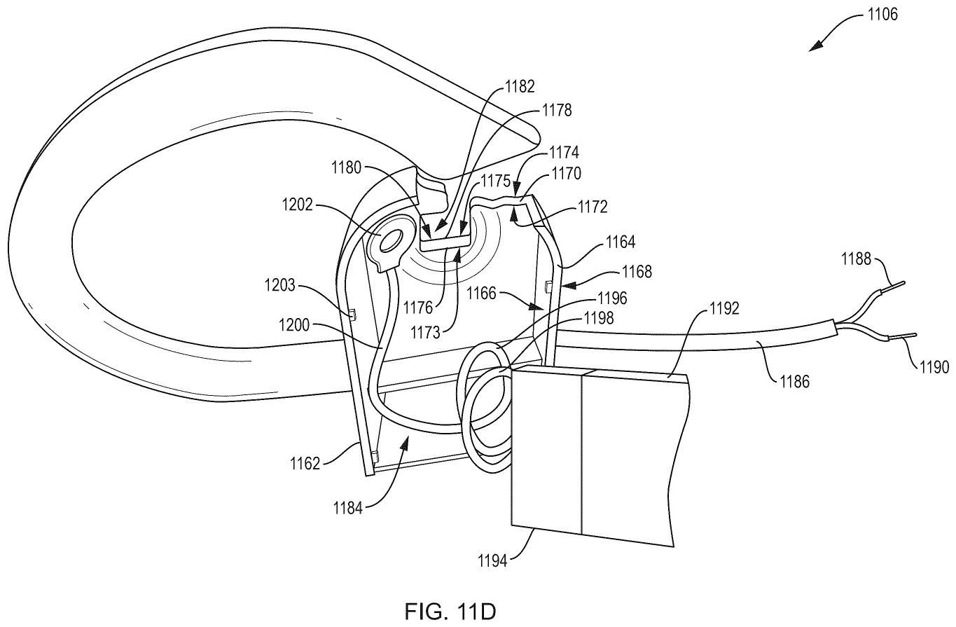

FIG. 11A shows, in cutaway perspective view, an alternative arrangement and embodiment of the invention including an assembly 1100 with a skin contacting electrode element 1102, a flange portion 1104 and an earpiece housing 1106.

The skin-contacting electrode element 1102 includes, for example, a body 1108 with a first generally annular portion 1110, a structural portion 1112 and a coupling column 1114. Annular portion 1110 includes electrode axial 1116 and electrode radial 1118 bearing surface regions. Structural portion 1112 includes a skin-contacting surface region 1113.

FIG. 11B shows, in perspective view, further details of the skin-contacting electrode element 1102, including body 1108 with, a first generally annular portion 1110, structural portion 1112 and coupling column 1114. As described above, annular portion 1110 includes electrode axial surface region 1116 and electrode radial surface region 1118.

In the illustrated embodiment, coupling column 1114 has the general aspect of a rectangular prism having a circumferential surface region 1120. A longitudinal axis 1122 is disposed generally normal to skin-contacting surface region 1113 of the structural portion 1112.

The illustrated coupling column 1114 has a circumferential edge region 1124 having a generally polygonal aspect that corresponds to a cross-section of the coupling column taken transverse to the longitudinal axis 1122.

The illustrated polygonal circumferential edge region 1124 is shown as generally square, with chamfered corners 1126. It will be appreciated by one of skill in the art, however, that the circumferential edge region (and a corresponding transverse section) of the coupling column 1114 will be rectangular, triangular, pentagonal, hexagonal, or have any other regular or irregular polygonal aspect deemed to be desirable in the context of a particular application of the invention.

In addition, the coupling column 1114 may, as shown, include one or more tapered surface portions, e.g., 1128, of circumferential surface region 1120 disposed adjacent an end 1130 of the coupling column 1114 that is relatively distal to surface region 1113.

In certain embodiments, the coupling column 1114 includes a longitudinal bore 1132. In certain embodiments of the invention, longitudinal bore 1132 includes a plurality of internal threads 1134 adapted to receive therewithin, for example, a machine screw. In other embodiments of the invention, longitudinal bore 1132 includes a surface adapted to receive a self-threading fastener such that internal threads are formed by insertion of the fastener. In still other embodiments of the invention, longitudinal bore 1132 will the arranged and configured to receive any of a variety of other fasteners such as are known, or become known, in the art.

In the illustrated embodiment, the skin-contacting electrode element 1102 has an electrically conductive surface. Accordingly, in a manner analogous to that shown above in FIG. 9 at 924, body 1108 of the skin-contacting electrode 1102 includes, for example, a synthetic polymer material. A layer of electroless nickel material is disposed upon, and supported by, a surface region of the polymer material. A further layer of electrolytically deposited silver is disposed upon, and supported by, a further surface region of the electrolytically deposited nickel material.

It will be appreciated by one of skill in the art that, as further discussed below, the electrically conductive nickel and silver materials will advantageously cover the entirety of the external surface of the skin-contacting electrode element 1102. Accordingly, for example, a surface region 1136 of structural portion 1112, disposed in generally parallel spaced relation to skin-contacting surface region 1113, is fully covered in the electrically conductive nickel and silver materials, as are circumferential surface region 1120 and a terminal surface region 1138 of coupling column 1114. In light of physical/mechanical continuity of the layers of material between the surface regions, electrical continuity will be present between terminal surface region 1138 and skin-contacting surface region 1113.

FIG. 11C shows, in cutaway perspective view, further aspects of flange portion 1104. As shown, flange portion 1104 includes a structural body 1140. Structural body 1140 includes a generally circular circumferential edge region 1142 consistent with the shape of the generally annular portion 1110. One of skill in the art will appreciate, however, that other forms are possible, and are to be employed where desirable within the scope of the invention.

Disposed between circular circumferential edge region 1142, and an annular groove 1144 is a flange axial surface region 1146 arranged to be disposed in contact with, and to support, electrode axial surface region 1116 of body 1108. A flange radial surface region 1147, is disposed inwardly of flange axial surface region 1146 and is arranged, in certain embodiments, to be disposed in contact with, and to support, electrode radial surface region 1118 of body 1108.

In the exemplary embodiment illustrated, the flange portion 1104 also includes a structural portion 1148 with a flange inner surface region 1150 disposed in substantially parallel spaced relation to a flange interface surface region 1152. An inner circumferential edge 1154 of flange inner surface region 1150 is disposed in substantially parallel spaced relation to an inner circumferential edge 1156 of flange interface surface region 1152, and an internal circumferential surface region 1158 is disposed between circumferential edges 1154 and 1156. The inner circumferential edges 1154, 1156 and internal circumferential surface region 1158 define a bore 1160 through structural portion 1148.

Inner circumferential edges 1154 and 1156 are generally symmetrical to one another as well as to the shape and dimensions of the cross-section (taken transverse to longitudinal axis 1122) of support column 1114, as discussed above. Accordingly, and as will be further discussed below, bore 1160 is sized as a clearance hole, and configured and adapted to receive a portion of support column 1114 therethrough.

FIG. 11D shows, in cutaway perspective view, further aspects of housing 1106. As shown, housing 1106 includes a structural body 1162. Structural body 1162 includes circumferential wall portions 1164 defined by respective inner 1166 and outer 1168 surface regions disposed in substantially parallel spaced relation with respect to one another.

Housing 1106 also includes a base portion 1170. Base portion 1170 has inner 1172 and outer 1174 surface regions disposed in substantially parallel spaced relation to one another. Surface regions 1172 and 1174 include respective inner circumferential edges 1176, 1178 which, together with an inner circumferential surface region 1180 define a bore 1182 through base portion 1170. As will be evident to the reader, the bore 1182 is shaped and sized similarly to bore 1160 and configured and adapted to receive a portion of support column 1114 therethrough. A portion of inner surface region 1172 disposed proximate to inner circumferential edge 1176 serves as a housing bearing surface region 1173.

A portion of outer surface region 1174 disposed proximate to inner circumferential edge 1178 serves as a housing interface surface region 1175.

Together, the surface regions 1166 and 1172 define a cavity 1184 within the body portion 1106. Cavity 1184 is configured and adapted to receive one end of a cable 1186 therewithin through a corresponding bore in wall portion 1164. Cable 1186 is similar to cable 810 of FIG. 8, and includes electrical conductors 1188 and 1190, similar to conductors 816 and 818.

Cavity 1184 is configured and adapted to have a battery, e.g., 1192 and/or electronics, e.g., 1194 disposed therewithin. The battery 1192 and/or electronics 1194 are, in an exemplary configuration, operatively coupled to respective terminal ends 1196, 1198 of electrical conductors 1188 and 1190.

Also operatively coupled to the battery 1192 and/or electronics 1194 is an exemplary signal conductor 1200. In the exemplary embodiment shown, conductor 1200 terminates at a connector 1202. The connector 1202 is, in respective embodiments, coupled to signal conductor 1200 by soldering, crimp in, or in any manner known or that becomes known in the art. Also, one of skill in the art will appreciate that the connector may be configured as a ring connector, as shown, as a fork connector, or in any other manner appropriate two the requirements of a particular instance or application of the invention.

Referring again to FIG. 11A, one of skill in the art will appreciate that the skin-contacting electrode element 1102 is assembled to flange portion 1104 and the body portion 1106 to form the completed assembly 1100. A threaded screw 1204, or other fastener, is disposed within bore 1132 aligned with longitudinal axis 1122 to establish integrity of the assembly. As shown, screw 1204 captures connector 1202, thereby providing an operative electrical connection between the battery 1192 and/or electronics 1194 through surface region 1138 (FIG. 11B) to skin-contacting surface 1113.

In light of the foregoing, one of skill in the art will appreciate that the polygonal section of the coupling column 1114, as disposed within apertures 1160 and 1182, will tend to prevent rotation of the skin-contacting electrode element as screw/fastener 1204 is rotated during assembly of the apparatus, and thereafter. Accordingly, the arrangement presented herewith serves to provide an improved electrical contact between signal conductor 1200 and surface region 1138 (as compared, for example, with the arrangement of FIG. 10), thereby enhancing the durability and reliability of the apparatus. As evident upon inspection of FIG. 11A, certain embodiments of the invention include a further washer 1206 disposed about fastener 1204 to enhance the structural integrity of the assembly.

It should be appreciated that the exemplary substantially square cross-section of coupling column 1114 illustrated in FIGS. 11A-11D is merely exemplary of a wide variety of cross-sections that will be beneficially applied in respective embodiments of the invention.

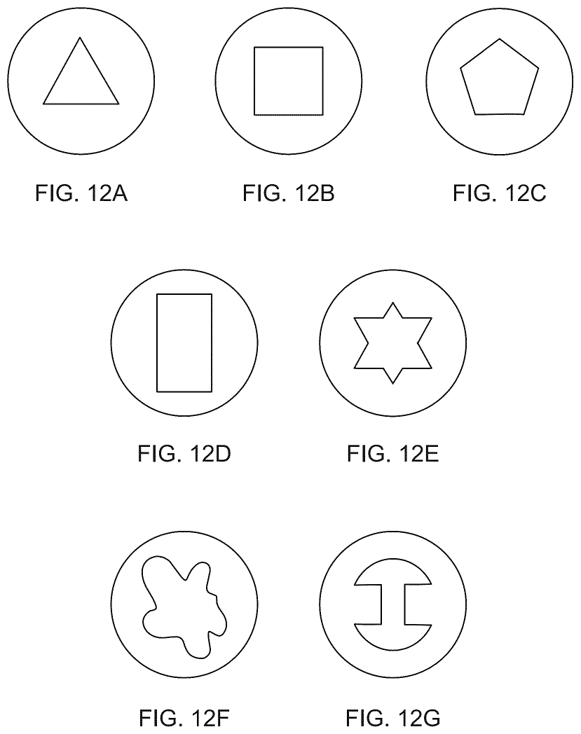

Accordingly, FIG. 12 shows in schematic plan view, a variety of alternative cross-sections that will be beneficially employed in various embodiments of the invention including, for example, triangular (FIG. 12A), square (FIG. 12B), pentagonal (FIG. 12C), rectangular (FIG. 12D), stellate (FIG. 12E), irregular (FIG. 12F) and combinations thereof (FIG. 12G).

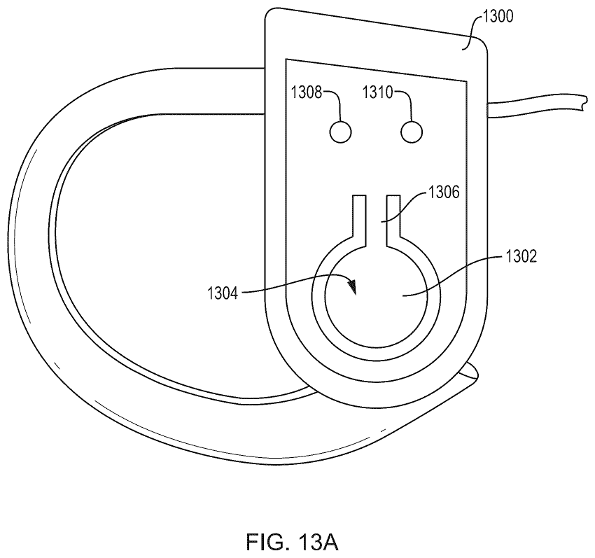

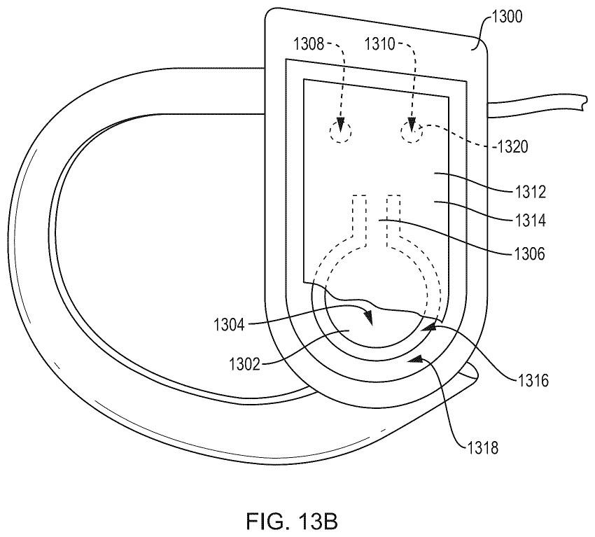

FIG. 13A shows, in schematic plan view, an exemplary cover element 1300 for a housing portion (like exemplary housing 1106). The cover portion 1300 is readily configured and adapted to complete the enclosure of, for example, a cavity 1184 within the housing portion.

The illustrated cover element 1300 shows a pushbutton actuator portion 1302 having a surface region 1304 to which finger pressure may be applied. Under such finger pressure, a flexure element 1306 is configured to flex, thereby allowing actuation of a switch apparatus within the cavity 1184 for operation and control of the earpiece device. The exemplary cover element 1300 also shows first 1308 and second 1310 apertures. By placing illuminating devices, such as, for example, light emitting diodes, below the first and second apertures, status indication may be provided to the user, indicating a condition of the device.

In certain embodiments of the invention, the cover element 1300 will include flanges (not shown) disposed inside of the cover element and including, for example, apertures configured to engage with detent protrusions (e.g., 1203 of FIG. 11D).

FIG. 13B shows, in perspective cutaway view, a sealing element 1312 for a signal generator earpiece prepared according to principles of the invention. Consistent with cover element 1300 of FIG. 13A, cover element 1300 shows a pushbutton actuator portion 1302 having a surface region 1304 to which finger pressure may be applied. The pushbutton actuator portion 1302 is coupled to the balance of the cover element 1300 by a flexure element 1306. As shown, a layer of flexible material 1314 spans an aperture 1316 between the pushbutton actuator portion 1302 and the balance of the cover element 1300.

In certain embodiments of the invention, flexible material 1314 is coupled by an adhesive to both surface region 1304 of actuator portion 1302 and to a further surface region 1318 outwardly of the aperture 1316. In other embodiments of the invention, flexible material 1314 is coupled to surface region 1318, but remains uncoupled from surface region 1304.

In still further embodiments of the invention, coupling between the flexible material 1314 and one or more of surfaces 1304 and 1318 is achieved by an attachment method such as, for example, ultrasonic welding, thermal welding, and intrinsic electrostatic forces. One of skill in the art will appreciate that any coupling method that is known, or becomes known in the art will be beneficially applied in a corresponding embodiment of the invention.

In a still further aspect of the invention, certain instances and embodiments of the flexible material 1314 will include a transparent and/or translucent region, e.g., 1320. In certain embodiments of the invention, this transparent or translucent region will encompass the entirety of the layer 1314. In other embodiments of the invention, the transparency and/or translucency of the region 1320 will be localized about, for example, one or both of apertures 1308 and 1310. In certain embodiments of the invention, the localized regions will include a colorant or other component or characteristic so that the color of illumination through the apertures 1308, 1310 is desirably affected by the color of the component or characteristic.

In still further aspects of the invention, the flexible material 1314 will include printed instructions, branding logos, and/or other indicia to desirably convey information to the user or another party.

In certain embodiments of the invention, the signal generator for auricular conchae includes an earpiece housing, a flange portion, and a skin-contacting electrode element for contacting a concha of a user. The skin contacting electrode element includes a coupling column. The coupling column has a longitudinal axis and a circumferential surface region. The circumferential surface region has a polygonal cross-section transverse to the longitudinal axis. The coupling column is adapted to be disposed through a polygonal bore of the earpiece housing so as to prevent rotation of the coupling column with respect to the earpiece housing. In some embodiments the polygonal cross-section is a regular polygon.

In certain embodiments of the invention a signal generator for auricular conchae includes an earpiece housing, a flange portion, and a skin-contacting electrode element for contacting a concha of a user. The skin contacting electrode element includes a coupling column, the coupling column has a longitudinal axis and a circumferential surface region. The circumferential surface region has a polygonal cross-section transverse to the longitudinal axis. The coupling column is adapted to be disposed through a polygonal bore of the flange portion so as to prevent rotation of the coupling column with respect to the flange portion.

While various embodiments of the disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

Although particular embodiments of the invention have been described in detail for purposes of illustration, various modifications and enhancements may be made without departing from the spirit and scope of the invention. The scope of the invention is accordingly defined by the following claims.

1. A signal generator for auricular conchae comprising:
an earpiece housing;
a flange portion; and
a skin-contacting electrode element for contacting a concha of a user, said skin contacting electrode element including a coupling column, said coupling column having a longitudinal axis and a circumferential surface region, said circumferential surface region exhibiting a polygonal cross-section transverse to said longitudinal axis, said coupling column being adapted to be disposed through a polygonal bore of said earpiece housing so as to prevent rotation of said coupling column with respect to said earpiece housing.

2. A signal generator for auricular conchae as defined in claim 1, wherein said coupling column includes a longitudinal bore disposed coaxially with said longitudinal axis, said longitudinal bore being adapted to receive a fastener therewithin, said fastener being adapted to couple said skin contacting electrode to said earpiece housing.

3. A signal generator for auricular conchae as defined in claim 2, wherein said fastener includes a threaded screw fastener.

4. A signal generator for auricular conchae as defined in claim 1, wherein said skin contacting electrode element includes a skin contacting surface region, wherein said coupling column includes a terminal surface region, and wherein said skin contacting electrode element includes an electrically conductive material disposed to electrically couple said terminal surface region to said skin contacting surface region.

5. A signal generator for auricular conchae as defined in claim 4, wherein said skin contacting electrode element includes a body portion and wherein said electrically conductive material includes a layer of metallic material deposited on a surface of said body portion.

6. A signal generator for auricular conchae as defined in claim 5, wherein said layer of metallic material covers an entire surface of said body portion.

7. A signal generator for auricular conchae as defined in claim 4, wherein said skin contacting electrode element includes a body portion and wherein said electrically conductive material includes a bulk material of said body portion.

8. A signal generator for auricular conchae as defined in claim 4, wherein a signal generator circuit is disposed within said earpiece housing, and wherein an output terminal of said signal generator circuit is electrically coupled to said terminal surface region of said skin contacting electrode element.

9. A signal generator for auricular conchae as defined in claim 8, wherein said output terminal of said signal generator circuit is electrically coupled to said terminal surface region of said skin contacting electrode element by a threaded fastener.

10. A signal generator for auricular conchae as defined in claim 8, wherein a further output terminal of said signal generator circuit is signalingly coupled to a further skin contacting electrode element of said signal generator so as to produce an electrical potential between said skin contacting electrode element and said further skin contacting electrode element.

11. A signal generator for auricular conchae comprising:
an earpiece housing;
a flange portion; and
a skin-contacting electrode element for contacting a concha of a user, said skin contacting electrode element including a coupling column, said coupling column having a longitudinal axis and a circumferential surface region, said circumferential surface region exhibiting a polygonal cross-section transverse to said longitudinal axis, said coupling column being adapted to be disposed through a polygonal bore of said flange portion so as to prevent rotation of said coupling column with respect to said flange portion.

12. A signal generator for auricular conchae as defined in claim 11, wherein said polygonal cross-section comprises a substantially square cross-section.

13. A signal generator for auricular conchae as defined in claim 11, wherein said coupling column further comprises:
a longitudinal bore disposed coaxial to said longitudinal axis, and
a fastener disposed within said longitudinal bore, said fastener being adapted to couple said skin contacting electrode to said earpiece housing.

14. A signal generator for auricular conchae as defined in claim 13, further comprising:
an electrical connector, said electrical connector being coupled to said skin contacting electrode element by said fastener.

15. A signal generator for auricular conchae as defined in claim 14, wherein said electrical connector comprises a ring connector.

16. A method of coupling an electronic circuit to a skin contacting electrode of a signal generator for auricular conchae, comprising:
providing an earpiece housing;
providing said skin-contacting electrode element including a coupling column, said coupling column having a longitudinal axis and a substantially polygonal circumferential surface region;
disposing said coupling column through a polygonal bore so as to prevent rotation of said coupling column with respect to said earpiece housing;
disposing an output terminal conductor of said electronic circuit in contact with a terminal end surface region of said skin contacting electrode element; and
disposing a fastener through said output terminal conductor into said coupling column to electrically couple said electronic circuit to said skin contacting electrode element and to mechanically couple said skin contacting electrode element to said earpiece housing.

17. A method of coupling an electronic circuit to a skin contacting electrode of a signal generator for auricular conchae as defined in claim 16, wherein said polygonal bore is disposed within said earpiece housing.

18. A method of coupling an electronic circuit to a skin contacting electrode of a signal generator for auricular conchae as defined in claim 16, wherein said polygonal bore is disposed within a flange of said earpiece housing.

19. A method of coupling an electronic circuit to a skin contacting electrode of a signal generator for auricular conchae as defined in claim 18, wherein said flange is integrally formed with said earpiece housing.

20. A method of coupling an electronic circuit to a skin contacting electrode of a signal generator for auricular conchae as defined in claim 16, wherein a conductive surface layer of said skin contacting electrode element is disposed to provide an electrical coupling between said terminal end surface region and a skin contacting surface region of said skin contacting electrode element.
